(19) United States
(12) Patent Application Publication
(10) Pub. No.: US 2020/0281675 A1
(43) Pub. Date: Sep. 10, 2020

(54) LOW COST DUAL CONSOLE TRAINING SYSTEM FOR ROBOTIC SURGICAL SYSTEM OR ROBOTIC SURGICAL SIMULATOR

(71) Applicant: Covidien LP, Mansfield, MA (US)

(72) Inventor: Dwight Meglan, Westwood, MA (US)

(21) Appl. No.: 16/797,004

(22) Filed: Feb. 21, 2020

Related U.S. Application Data
(60) Provisional application No. 62/813,413, filed on Mar. 4, 2019.

(51) Int. Cl. A61B 34/37

(52) U.S. Cl. CPC A61B 34/37; A61B 2017/00544

(57) ABSTRACT

A dual user console for a robotic surgical system includes first and second user consoles. The first and user consoles each include an arm, an actuator, and a manifold. Each arm has a joint and the actuator is disposed at the joint. The actuator includes pneumatic and hydraulic cylinders that are each configured to actuate the arm about the joint and to be back driven by movement of the arm about the joint. The manifold is coupled to the pneumatic and hydraulic cylinders. The manifolds are in communication with one another such that movements of the arm of the first console about its joint are mirrored to movements of the arm of the second user console about its joint and movements of the arm of the second user console about its joint are mirrored to movements of the arm of the first user console about its joint.

### CROSS-REFERENCE TO RELATED APPLICATIONS

This application claims the benefit of and priority to U.S. Provisional Patent Application Ser. No. 62/813,413 filed Mar. 4, 2019, the entire disclosure of which is incorporated by reference herein.

### BACKGROUND

Robotic surgical systems have been used in minimally invasive medical procedures. During a medical procedure, the robotic surgical system is controlled by a surgeon interfacing with a user interface. The user interface allows the surgeon to manipulate an end effector of a surgical instrument that acts on a patient. The user interface includes an input controller or handle that is moveable by the surgeon to control the robotic surgical system.

Different robotic surgical systems exist in the market each with different controls and displays. As a surgeon moves from one robotic surgical system to another, the surgeon must familiarize themselves with the controls and the displays of the particular robotic surgical system. In addition, during a surgical procedure, each system may have different alerts or alarms to indicate a condition of the robotic surgical system or the patient which may delay recognition of the alarm to a surgeon not familiar with the particular robotic surgical system.

As a training surgeon learns, becomes familiar with, or trains to use a robotic surgical system, the training surgeon may use a dual console user interface with an experienced surgeon to control the surgical robot. Generally, dual console user interfaces are configured to provide handoff control between an experienced or instructor surgeon and the training surgeon. Traditional dual console user interfaces are expensive and necessitate a significant amount of space as two full sized user interfaces are required.

### SUMMARY

This disclosure relates generally to a dual console user interface for a robotic surgical system or robotic surgical simulator that requires less space than a traditional dual console user interface and/or allows for shared control of the surgical robot. The shared control may be particularly useful during the training of a surgeon as the trainee can feel the same sensations that the experienced surgeon is feeling during the surgical procedure.

In an aspect of the present disclosure, a dual user console for a robotic surgical system includes a first user console and a second user console. The first user console includes a first arm, a first actuator, and a first manifold. The first arm has a first joint and the first actuator is disposed at the first joint. The first actuator includes a first pneumatic cylinder and a first hydraulic cylinder that are each configured to actuate the first arm about the first joint and to be back driven by movement of the first arm about the first joint. The first manifold is coupled to the first pneumatic and hydraulic cylinders. The second user console includes a second arm, a second actuator, and a second manifold. The second arm has a second joint and the second actuator is disposed at the second joint. The second actuator includes a second pneumatic cylinder and a second hydraulic cylinder that are each configured to actuate the second arm about the second joint and to be back driven by movement of the second arm about the second joint. The second manifold is coupled to the second pneumatic and hydraulic cylinders. The first manifold is in communication with the second manifold such that movements of the first arm about the first joint are mirrored to movements of the second arm about the second joint and movements of the second arm about the second joint are mirrored to movements of the first arm about the first joint.

In aspects, the first user console includes a first pneumatic line and a first hydraulic line. The first pneumatic line may have a first end that is coupled to the first pneumatic cylinder and a second end that is coupled to the first manifold. The first hydraulic line may have a first end that is coupled to the first hydraulic cylinder and a second end that is coupled to the first manifold.

In some aspects, the first manifold is disposed on the first arm. The first manifold may include a first console manifold and a first arm manifold. The first arm manifold may be disposed on the first arm. The dual console may include a pneumatic manifold interconnect that extends between the first console manifold and the first arm manifold. The dual console may include a hydraulic manifold interconnect that extends between the first console manifold and the first arm manifold.

In certain aspects, the dual console includes a pneumatic console interconnect that extends between the first manifold and the second manifold to pneumatically interconnect the first and second manifolds. The dual console may include a hydraulic console interconnect that extends between the first manifold and the second manifold to hydraulically couple the first and second manifolds.

In particular aspects, the first manifold is in wired or wireless communication with the second manifold. The first manifold may be configured to transmit positions of the first pneumatic and hydraulic cylinders to the second manifold such that the second manifold actuates the second pneumatic and hydraulic cylinders to mirror the positions of first pneumatic and hydraulic cylinders. The second manifold may be configured to transmit positions of the second pneumatic and hydraulic cylinders to the first manifold such that the first manifold actuates the first pneumatic and hydraulic cylinders to mirror the positions of the second pneumatic and hydraulic cylinders.

In some embodiments, the first user console includes a third arm that has a third joint and a third actuator disposed at the third joint. The second user console may include a fourth arm that has a fourth joint and a fourth actuator disposed at the first joint. The third actuator is coupled to the first manifold and the fourth actuator is coupled to the second manifold. The first manifold may be in communication with the second manifold such that movements of the third arm about the third joint arm mirrored to movements of the fourth arm about the fourth joint and movements of the fourth arm about the fourth joint are mirrored to movements of the third arm about the third joint.

In another aspect of the present disclosure, a robotic surgical system includes a processing unit, a surgical robot having a first robot arm, and a dual user console having a first and second user console. The first user console is in communication with the processing unit and is configured to transmit input signals to the processing unit. The processing unit is configured to transmit control signals to the surgical robot in response to the input signals such that the surgical robot manipulates the first robot arm in response to the control signals. The first user console includes a first arm, a first actuator, and a first manifold. The first arm has a first joint and the first actuator is disposed at the first joint. The first actuator includes a first pneumatic cylinder and a first hydraulic cylinder that are each configured to actuate the first arm about the first joint and to be back driven by movement of the first arm about the first joint. The first manifold is coupled to the first pneumatic and hydraulic cylinders. The second user console includes a second arm, a second actuator, and a second manifold. The second arm has a second joint and the second actuator is disposed at the second joint. The second actuator includes a second pneumatic cylinder and a second hydraulic cylinder that are each configured to actuate the second arm about the second joint and to be back driven by movement of the second arm about the second joint. The second manifold is coupled to the second pneumatic and hydraulic cylinders. The first manifold is in communication with the second manifold such that movements of the first arm about the first joint are mirrored to movements of the second arm about the second joint and movements of the second arm about the second joint are mirrored to movements of the first arm about the first joint.

In aspects, the first user console includes a third arm that has a third joint and a third actuator disposed at the third joint. The second user console may include a fourth arm that has a fourth joint and a fourth actuator disposed at the fourth joint. The first user console may include a third manifold that is disposed on the third arm and the second user console may include a fourth manifold that is disposed on the fourth arm. The third manifold is in communication with the fourth manifold such that movements of the third arm about the third joint are mirrored to movements of the fourth arm about the fourth joint. Movements of the fourth arm about the fourth joint may be mirrored to movements of the third arm about the third joint.

In some aspects, the first user console includes a first console manifold in direction communication with the first and third manifolds and the second user console includes a second console manifold that is in direction communication with the second and fourth manifolds. The first and second console manifolds may be in direct communication with one another such that the first and second manifolds and the third and fourth manifolds are in communication with one another. The dual user console may include pneumatic and hydraulic console interconnects. The pneumatic console interconnect includes a first discreet pneumatic channel that is configured to couple the first and second actuators and a second discreet pneumatic channel that is configured to couple the third and fourth actuators. The hydraulic console interconnect may include a first discreet hydraulic channel that is configured to couple the first and second actuators and a second discreet hydraulic channel that is configured to couple the third and fourth actuators.

In another aspect of the present disclosure, a method of controlling a surgical robot or a surgical simulator includes manipulating a first arm of a first user console about a first joint such that a first actuator associated with the first joint transmits first pneumatic and hydraulic signals to a second actuator associated with a second joint of a second arm of a second user console to actuate the second arm to mirror the manipulation of the first arm and manipulating the second arm of the second user console about the second joint such that the second actuator transmits second pneumatic and hydraulic singles to the first actor is associated with the first joint to actuate the first arm to mirror the manipulation of the second arm.

In aspects, manipulating the first arm that includes transmitting the first pneumatic and hydraulic signals to a first manifold of the first user console which transmits the first pneumatic and hydraulic signals to a second manifold of the second user interface. The first manifold may transmit the first pneumatic and hydraulic signals to the second manifold includes wirelessly transmitting the first pneumatic and hydraulic signals.

In some aspects, the method includes selecting a preprogramed surgical technique such that a processing unit of the surgical robot or the surgical simulator transmits control signals to the first manifold. The first manifold may transmit pneumatic and hydraulic signals to the first actuator in response to the control signals such that the first actuator actuates the first arm about the first joint.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic view of a robotic surgical system in accordance with the present disclosure including a dual user consoles, a surgical robot, and a shared control system;

FIG. 2 is a perspective view of a control arm of one of the user consoles of FIG. 1;

FIG. 3 a top view of an actuator of the control arm of FIG. 2;

FIGS. 4A and 4B are cross-sectional views of taken along the section line 4-4 of FIG. 2;

FIG. 5 is a schematic view of the dual user consoles of FIG. 1 with another shared control system provided in accordance with the present disclosure; and

FIG. 6 is a schematic view of the dual user consoles of FIG. 1 with another shared control system provided in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a surgeon, a nurse, or any other care provider and may include support personnel. Throughout this description, the term “proximal” refers to the portion of the device or component thereof that is closer to the clinician and the term “distal” refers to the portion of the device or component thereof that is farther from the clinician.

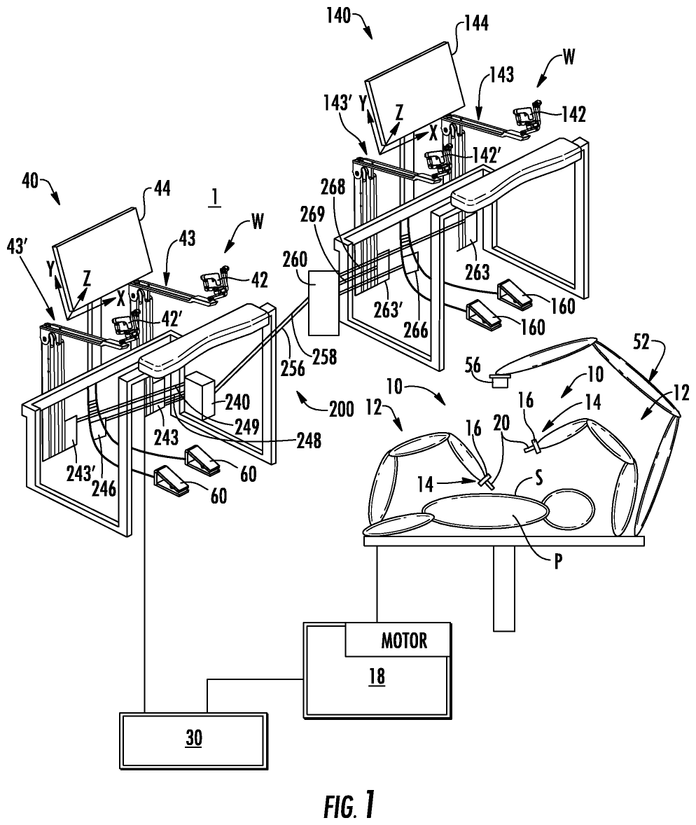

Referring to FIG. 1, a robotic surgical system 1 in accordance with the present disclosure is shown generally as a surgical robot 10, a processing unit 30, a master user console 40, and a slave user console 140. The surgical robot 10 generally includes linkages 12 and a robot base 18. The linkages 12 moveably support an end effector or tool 20 which is configured to act on tissue. The linkages 12 may be in the form of arms each having an end 14 that supports the end effector or tool 20 which is configured to act on tissue. In addition, the ends 14 of the linkages 12 may include an imaging device 16 for imaging a surgical site “S”. The master user console 40 is in communication with robot base 18 through the processing unit 30.

The master user console 40 includes a display device 44 which is configured to display three-dimensional images. The display device 44 displays three-dimensional images of the surgical site “S” which may include data captured by imaging devices 16 positioned on the ends 14 of the linkages 12 and/or include data captured by imaging devices that are positioned about the surgical theater (e.g., an imaging device positioned within the surgical site “S”, an imaging device positioned adjacent the patient “P”, imaging device 56 positioned at a distal end of an imaging arm 52). The imaging devices (e.g., imaging devices 16, 56) may capture visual images, infra-red images, ultrasound images, X-ray images, thermal images, and/or any other known real-time images of the surgical site “S”. The imaging devices transmit captured imaging data to the processing unit 30 which creates three-dimensional images of the surgical site “S” in real-time from the imaging data and transmits the three-dimensional images to the display device 44 for display.

The master user console 40 also includes input handles 42, 42′ which are supported on control arms 43, 43′ which allow a clinician to manipulate the surgical robot 10 (e.g., move the linkages 12, the ends 14 of the linkages 12, and/or the tools 20). Each of the input handles 42, 42′ is in communication with the processing unit 30 to transmit control signals thereto and to receive feedback signals therefrom. Additionally or alternatively, each of the input handles 42, 42′ may include input devices (not explicitly shown) which allow the surgeon to manipulate (e.g., clamp, grasp, fire, open, close, rotate, thrust, slice, etc.) the tools 20 supported at the ends 14 of the linkages 12.

Each of the input handles 42, 42′ is moveable through a predefined workspace to move the ends 14 of the linkages 12, e.g., tools 20, within a surgical site “S”. The three-dimensional images on the display device 44 are orientated such that the movement of the input handles 42, 42′ moves the ends 14 of the linkages 12 as viewed on the display device 44. The three-dimensional images remain stationary while movement of the input handles 42, 42′ is scaled to movement of the ends 14 of the linkages 12 within the three-dimensional images. To maintain an orientation of the three-dimensional images, kinematic mapping of the input handles 42, 42′ is based on a camera orientation relative to an orientation of the ends 14 of the linkages 12. The orientation of the three-dimensional images on the display device 44 may be mirrored or rotated relative to the view captured by the imaging devices 16, 56. In addition, the size of the three-dimensional images on the display device 44 may be scaled to be larger or smaller than the actual structures of the surgical site permitting a clinician to have a better view of structures within the surgical site “S”. As the input handles 42, 42′ are moved, the tools 20 are moved within the surgical site “S” as detailed below. Movement of the tools 20 may also include movement of the ends 14 of the linkages 12 which support the tools 20.

For a detailed discussion of the construction and operation of a robotic surgical system 1, reference may be made to U.S. Pat. No. 8,828,023, the entire contents of which are incorporated herein by reference.

The master user console 40 further includes one or more foot pedals 60 that can be used to control various aspects of the robotic surgical system 1. For example, the foot pedal 60 may be selectively associated with an input handle, e.g., input handle 42, to actuate a tool 20 associated with the respective input handle. Additionally or alternatively, the foot pedal 60 may be associated with a camera, e.g., camera 56, to move the camera about the surgical site “S”. For a detailed discussion of suitable foot pedals, reference may be made to U.S. Provisional Patent Application Ser. No. 62/510,502, filed May 24, 2017, entitled “PEDAL CONTROL FOR ROBOTIC SURGICAL SYSTEMS,” and U.S. Provisional Patent Application Ser. No. 62/566,100, filed Sep. 8, 2017, entitled “HIGH PRECISION INSTRUMENT CONTROL MODE FOR ROBOTIC SURGICAL SYSTEMS,” the entire contents of each of the above applications are hereby incorporated by reference.

The slave user console 140 is similar to the master user console 40 detailed above and only the differences will be detailed herein for brevity. The slave user console 140 is in communication with the master user console 40 via a shared control system 200 such that the controls of the slave user console 140 are not in direct communication with the processing unit 30. It is contemplated that while the controls of the slave user console 140 are not in direct communication with the processing unit 30 that some elements, e.g., warning lights, indicator lights, and other audible or visual feedback elements, of the slave user console 140 may be in direct communication with the processing unit 30. In addition, the display 144 of the slave user console 140 may be in direct communication with the processing unit 30 to receive images from the processing unit 30 and to send signals to the processing unit 30.

As detailed above and shown in FIG. 1, the master and slave user interfaces 40, 140 are in operable communication with the surgical robot 10 to perform a surgical procedure on a patient “P”; however, it is envisioned that the master and slave user interfaces 40, 140 may be in operable communication with a surgical simulator (not shown) to virtually actuate a robot system and/or tool in a simulated environment. For example, the robotic surgical system 1 may have a first mode where the master and slave user interfaces 40, 140 are coupled to actuate the surgical robot 10 and a second mode where the master and slave user interfaces 40, 140 are coupled to the surgical simulator to virtually actuate a surgical robot. The surgical simulator may be a standalone unit or be integrated into the processing unit 30. The surgical simulator virtually responds to a clinicians interfacing with the master and slave user interfaces 40, 140 by providing visual, audible, force, and/or haptic feedback to clinicians through the master and slave user interfaces 40, 140. For example, as a clinician interfaces with the input device handles 42, 142, the surgical simulator moves representative tools that are virtually acting on tissue at a simulated surgical site. It is envisioned that the surgical simulator may allow a clinician to practice a surgical procedure before performing the surgical procedure on a patient. In addition, the surgical simulator may be used to train a clinician on a surgical procedure. Further, the surgical simulator may simulate “complications” during a proposed surgical procedure to permit a clinician to plan a surgical procedure.

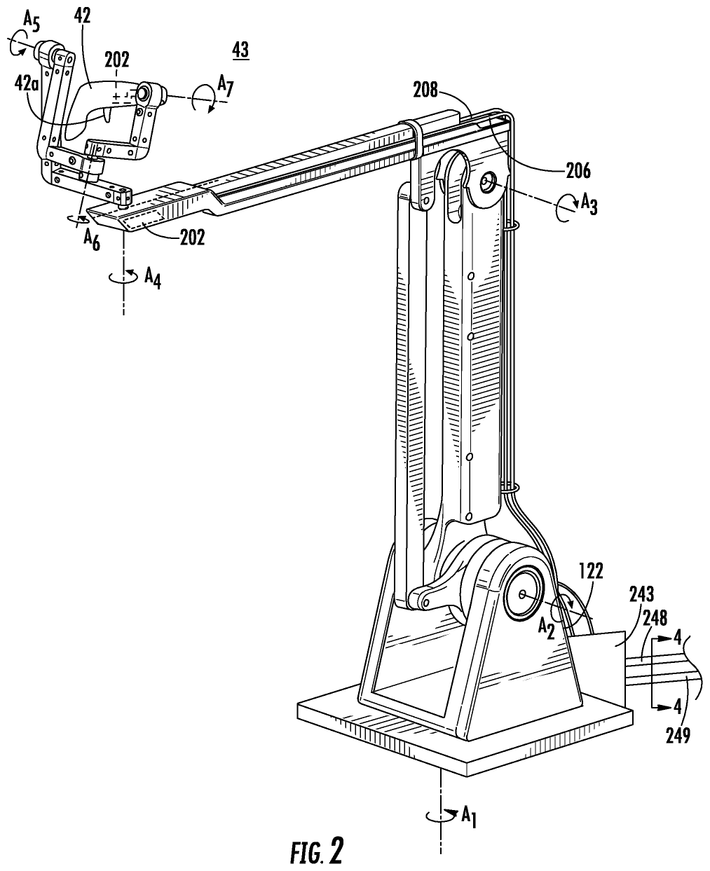

Referring now to FIGS. 1 and 2, the shared control system 200 is provided in accordance with the present disclosure to synchronize the controls of the master user console 40 and the controls of the slave user console 140. It will be appreciated, that the term master and slave are used with respect to how the user consoles 40, 140 are related to the connection with the processing unit 30 and that movements of either one of the user consoles 40, 140 may be translated to the other user console 40, 140 as detailed below.

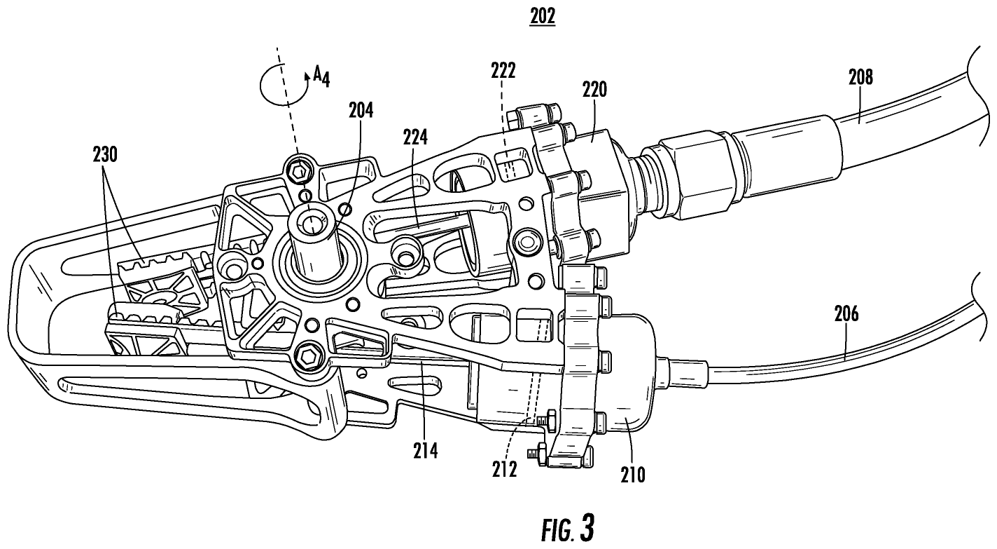

With additional reference to FIG. 3, the shared control system 200 includes an actuator 202 at each joint, e.g., fourth axis of rotation A₄, of the control arm 43. The actuators 202 are hybrid actuators including a spindle 204, a pneumatic line 206, and a hydraulic line 208. The pneumatic line 206 functions as a preloaded spring and damping system for rotation of the spindle 204. The hydraulic line 208 provides torque to rotate the spindle 204. The spindles 204 of the actuators 202 are capable of being driven by the pneumatic and hydraulic lines 206, 208 and are capable of being back driven.

The pneumatic line 206 is coupled to a cylinder 210 to rotate the spindle 204. The cylinder 210 includes a diaphragm 212 that is coupled to a rod 214. The diaphragm 212 moves back and forth within the cylinder to rotate the spindle 204. The rod 214 is coupled to a gear system 230 that converts linear actuation of the rod 214 into rotation of the spindle 204.

Similarly, the hydraulic line 206 is coupled to a cylinder 220 to rotate the spindle 204. The cylinder 220 includes a diaphragm 222 that is coupled to a rod 224. The diaphragm 222 moves back and forth within the cylinder to rotate the spindle 204. The rod 224 is coupled to the gear system 230 that converts linear actuation of the rod 214 into rotation of the spindle 204.

Referring back to FIG. 2, the handle 42 includes one or more actuator(s) 202 associated with a control interface of the handle 42, e.g., lever or trigger 42a. The actuator 202 for the handle 42 may include a spindle 204 to rotate the respective control interface or the gear system 230 (FIG. 3) may linearly actuate a respective control interface, e.g., trigger 42a. It will be appreciated that each actuator 202 of the handle 42 includes a pneumatic line and a hydraulic line similar to pneumatic and hydraulic lines 206, 208.

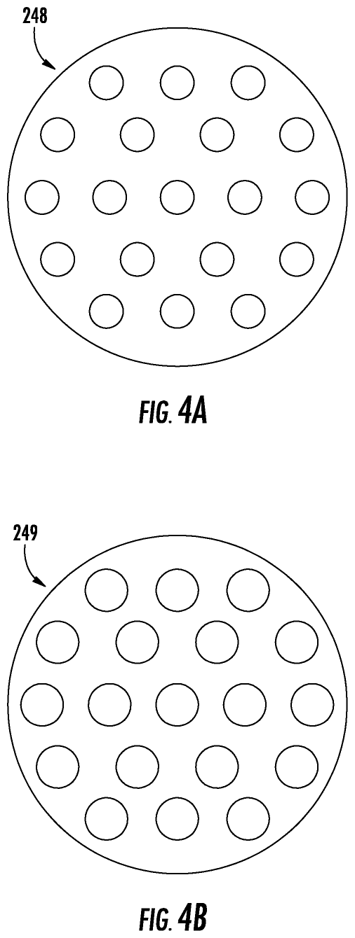

With continued reference to FIG. 2, the pneumatic and hydraulic lines 206, 208 of each actuator 202 extend from the respective joint or control interface, e.g., fourth axis of rotation A₄, to a master manifold 240 of the master user console 40. The master manifold 240 includes a connection for each of the pneumatic and hydraulic lines 206, 208 from the master user console 40 to provide a single point of connection for each of the pneumatic and hydraulic lines 206, 208 off of the master user console 40. It is contemplated that each arm 43, 43′ and/or the foot pedals 60 may include an arm or pedal manifold 243, 243′, 246 that provides a single point of connection for the respective arm 43, 43′ or foot pedals 60 to reduce the number of loose lines extending between a particular arm 43, 43′ or foot pedals 60 and the master manifold 240. It will be appreciated that each arm or pedal manifold 243, 243′, 246 is coupled to the master manifold 240 by a pneumatic manifold interconnect line 248 and a hydraulic manifold interconnect line 249. Each of the pneumatic and hydraulic manifold interconnect lines 248, 249 includes a discreet internal channel for each of the pneumatic and hydraulic lines, e.g., pneumatic and hydraulic lines 206, 208, that enter the respective arm or pedal manifold 243, 243′, 246 as shown in FIGS. 4A and 4B. While the pneumatic and hydraulic manifold interconnect lines 248, 249 are shown as a single line with a single connection to the master manifold 240 and the respective arm or pedal manifold 243, 243′, 246, the pneumatic and hydraulic manifold interconnect lines 248, 249 may be a bundle of lines with a separate connector for each line.

Referring briefly back to FIG. 1, the master manifold 240 is coupled to a slave manifold 260 by a pneumatic console interconnect line 256 and a hydraulic console interconnect line 258. The pneumatic and hydraulic console interconnect lines 256, 258 include a discreet channel for each of the pneumatic and hydraulic lines, e.g., pneumatic and hydraulic lines 206, 208, that are coupled to the master manifold 240. While the pneumatic and hydraulic console interconnect lines 256, 258 are shown as a single line with a single connection to each of the master and slave manifolds 240, 260, the pneumatic and hydraulic console interconnect lines 256, 258 may be a bundle of lines with a separate connector for each line or group of lines, e.g., a separate connector for an arm group of lines.

The slave user console 140 includes actuators 202 at joints and control interfaces of the first and second arms 143, 143′ and pedals 160 that correspond to the actuator 202 of the master user console 40. The shared control system 200 interconnects the pneumatic and hydraulic lines from each of the actuators 202 of the master user console 40 with a corresponding actuator 202 of the slave user console 140 such that as the joint or the control interface of the master user console 40 is manipulated, the shared control system 200 manipulates the corresponding joint of the slave user console 140 in a similar manner. Specifically, as the joint or the control interface of the master user console 40 back drives the associated actuator 202, the corresponding joint or control interface of the slave user console 140 drives the corresponding actuator 202 such that the corresponding joint or control interface of the slave user console 140 mirrors the movement of the joint or the control interface of the master user console 40. It will be appreciated that movement of a joint or control interface of the slave user console 140 may be mirrored to a joint or control interface of the master user console 40 in a similar manner.

By mirroring movements between the master user console 40 and the slave user console 140, movements of the surgical robot 10 (FIG. 1) can be controlled by either of the master or slave user consoles 40, 140 simultaneously. In addition, movements of one of the user consoles, e.g., master user console 40, may be controlled by a teaching clinician such that a learning clinician can experience movements through the other one of the user console, e.g., slave user console 140. Similarly, a learning clinician can use one of the user consoles, e.g., slave user console 140, to perform a surgical procedure with the surgical robot 10 while a teaching clinician observes the surgical procedure from the other user console, e.g., master user console 40, to provide guidance and/or physical input to assist the learning clinician.

As detailed above, the master and slave user consoles 40, 140 may be used with a surgical simulator instead of a surgical robot. When the master and slave user consoles 40, 140 are used with a surgical simulator, the teaching and learning clinicians can perfect a skill or an entire surgical procedure without acting on a patient.

Referring now to FIG. 5, another shared control system 300 is disclosed in accordance with the present disclosure. The shared control system 300 includes the actuators 202 (FIG. 2) and the pneumatic and hydraulic lines associated with each of the actuators 202 of the master and slave user consoles 40, 140, e.g., pneumatic and hydraulic lines 206, 208. The shared control system 300 includes a master manifold 340 and a slave manifold 360. The master manifold 340 and the slave manifold 360 receive the pneumatic and hydraulic lines from the actuators 202 in a similar manner to the master and slave manifolds 240, 260 detailed above.

The master manifold 340 and the slave manifold 360 include a pneumatic fluid actuator 306 associated with each pneumatic line, e.g., pneumatic line 206, and a hydraulic fluid actuator 308 associated with each hydraulic line 208. Each of the pneumatic and hydraulic fluid actuators 306, 308 are configured to remotely drive the associated actuator 202 and be back driven by the associated actuator 202. Each pneumatic and hydraulic fluid actuator 306, 308 includes a sensor 310 configured to determine the position of the diaphragm 212, 222 (FIG. 3) of the associated actuator 202.

The master manifold 340 includes a master controller 342 that is in communication with each of the pneumatic and hydraulic fluid actuators 306, 308 of the master manifold 340 and each of the sensors 310. Similarly the slave manifold 360 includes a slave controller 362 that is in communication with each of the pneumatic and hydraulic fluid actuators 306, 308 of the slave manifold 340. The master controller 342 is in communication with the slave controller 362 to control the corresponding pneumatic and hydraulic fluid actuators 306, 308 of the respective manifold 340, 360 such that the corresponding actuators 202 are mirrored between the master and slave user interfaces 40, 140.

The master and slave controllers 342, 362 may be in direct electrical communication with one another, e.g., be directly wired to one another, or may be in wireless communication with one another. Additionally or alternatively, the master and slave controllers 342, 362 may be in communication, either directly or wirelessly, with the processing unit 30 (FIG. 1). Each of the controllers 342, 362 converts the positions of the diaphragms 212, 222 associated with the pneumatic and hydraulic lines 206, 208 to electrical signals and transmits the positions to the controller 342, 362 of a corresponding manifold 340, 360 which may reduce the need for multiple pneumatic and hydraulic interconnect lines to run between manifolds and/or the user consoles. The reduction in interconnect lines may reduce the cost of the robotic surgical system. In addition, the reduction in interconnect lines may reduce the number of lines within the surgical space and increase the safety and/or the flexibility of the robotic surgical system.

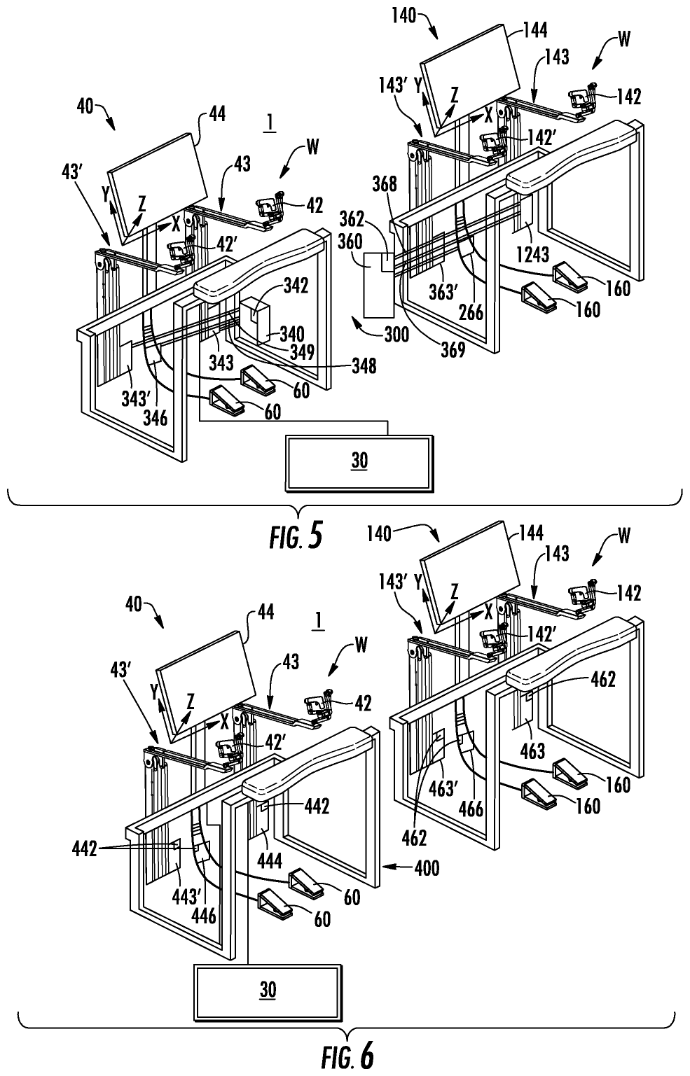

With reference to FIG. 6, another shared control system 400 is disclosed in accordance with the present disclosure. The shared control system 400 includes the actuators 202 and the pneumatic and hydraulic lines associated with each of the actuators 202 of the master and slave user consoles 40, 140, e.g., pneumatic and hydraulic lines 206, 208. The shared control system 400 includes master arm manifolds 443, 443′, a master foot manifold 446, slave arm manifolds 463, 463′, and a slave foot manifold 466. The master manifolds 443,443′, 446 and slave manifolds 463, 463′, 466 receive the pneumatic and hydraulic lines from the actuators 202 in a similar manner to the master arm and foot manifolds 243, 243′, 246 and the slave manifolds 463, 463′, 466 receive the pneumatic and hydraulic lines form the actuators 202 in a similar manner to the slave arm and foot manifolds 263, 263′, 266 detailed above.

Each of the master arm and foot manifolds 443, 443′, 446 is similar to the master manifold 340 detailed above with a pneumatic and hydraulic fluid actuator 406, 408 associated with each of the pneumatic and hydraulic lines 206, 208, respectively. In addition, each of the slave arm and foot manifolds 463, 463′, 466 is similar to the slave manifold 360 detailed above with a pneumatic and hydraulic fluid actuator 406, 408 associated with each of the pneumatic and hydraulic lines 206, 208, respectively.

Each of the master and slave arm and foot manifolds 443, 443′, 446, 463, 463′, 466 includes a controller 442, 462 that is in communication with each of the pneumatic and fluid actuators 406, 408 and sensors 410 of the respective manifold 443, 443′, 446, 463, 463′, 466. The controller 442 of the master arm manifold 443 is in communication with the controller 462 of the slave arm manifold 463, the controller 442 of the master arm manifold 443′ is in communication with the controller 462 of the slave arm manifold 463′, and the controller 442 of the master foot manifold 446 is in communication n with the controller 462 of the slave foot manifold 466 such that the actuators 202 of the slave user console 140 mirror the corresponding actuators 202 of the master user console 40 and vice versa as detailed above.

The controllers 442, 462 may be in wired or wireless communication with one another and/or with the processing unit 30 (FIG. 1). Each of the controllers 442, 462 converts the positions of the diaphragms 212, 222 associated with the pneumatic and hydraulic lines 206, 208 to electrical signals and transmits the positions to the controller 442, 462 of a corresponding manifold 443, 443′, 446, 463, 463′, 466 may reduce the need for multiple pneumatic and hydraulic interconnect lines to run between manifolds and/or the user consoles. The reduction in interconnect lines may reduce the cost of the robotic surgical system. In addition, the reduction in interconnect lines may reduce the number of lines within the surgical space and increase the safety and/or the flexibility of the robotic surgical system.

The shared control systems 300 and 400 may be used with a programmed surgical technique or procedure to teach the technique or the procedure to a clinician. The processing unit 30 may be preprogrammed with one or more surgical techniques or procedures such that a clinician interfacing with one of the master or slave user consoles 40, 140 may select a preprogramed technique or procedure. The processing unit 30 then transmits the movements of the technique or the procedure to the manifold or manifold(s), e.g., master manifold 340, of the user console while transmitting images of the simulated procedure to the displays associated with the user consoles, e.g., displays 44, 144 (FIG. 1).

The shared control systems 300 and 400 may be used to program a surgical technique or procedure such that the technique or produce can be taught to clinicians as detailed above. Specifically, when being used during a simulated or actual surgical procedure, a clinician may select a recording option such that the processing unit 30 captures images from the display, e.g., display 44, and positions of the actuators 202 from the manifold(s), e.g., master manifold 340, during a simulated or actual surgical procedure such that the images and positions of the actuators 202 can be transmitted as a preprogramed technique or procedure as detailed above.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A dual user console for a robotic surgical system, the dual user console comprising:
a first user console including:
a first arm having a first joint;
a first actuator disposed at the first joint and including a first pneumatic cylinder and a first hydraulic cylinder, each of the first pneumatic and hydraulic cylinders configured to actuate the first arm about the first joint and to be back driven by movement of the first arm about the first joint; and
a first manifold coupled to the first pneumatic cylinder and the first hydraulic cylinder; and
a second user console including:
a second arm having a second joint;
a second actuator disposed at the second joint and including a second pneumatic cylinder and a second hydraulic cylinder, each of the second pneumatic and hydraulic cylinders configured to actuate the second arm about the second joint and to be back driven by movement of the second arm about the second joint; and
a second manifold coupled to the second pneumatic cylinder and the second hydraulic cylinder,
wherein the first manifold is in communication with the second manifold such that movements of the first arm about the first joint are mirrored to movements of the second arm about the second joint and movements of the second arm about the second joint are mirrored to movements of the first arm about the first joint.

2. The dual console according to claim 1, wherein the first user console includes a first pneumatic line and a first hydraulic line, the first pneumatic line having a first end coupled to the first pneumatic cylinder and a second end coupled to the first manifold, the first hydraulic line having a first end coupled to the first hydraulic cylinder and a second end coupled to the first manifold.

3. The dual console according to claim 1, wherein the first manifold is disposed on the first arm.

4. The dual console according to claim 1, wherein the first manifold includes a first console manifold and a first arm manifold.

5. The dual console according to claim 4, wherein the first arm manifold is disposed on the first arm.

6. The dual console according to claim 4, further comprising a pneumatic manifold interconnect extending between the first console manifold and the first arm manifold, and a hydraulic manifold interconnect extending between the first console manifold and the first arm manifold.

7. The dual console according to claim 1, further comprising a pneumatic console interconnect extending between the first manifold and the second manifold, and a hydraulic console interconnect extending between the first manifold and the second manifold, the pneumatic console pneumatically coupling the first and second manifolds, the hydraulic console interconnect hydraulically coupling the first and second manifolds.

8. The dual console according to claim 1, wherein the first manifold is in wireless or wireless communication with the second manifold, the first manifold configured to transmit positions of the first pneumatic and first hydraulic cylinders to the second manifold such that the second manifold actuates the second pneumatic and second hydraulic cylinders to mirror the positions of first pneumatic and hydraulic cylinders.

9. The dual console according to claim 8, wherein the second manifold is configured to transmit positions of the second pneumatic and hydraulic cylinders to the first manifold such that the first manifold actuates the first pneumatic and hydraulic cylinders to mirror the positions of the second pneumatic and hydraulic cylinders.

10. The dual console according to claim 1, wherein the first user console includes a third arm having a third joint and a third actuator disposed at the third joint, wherein the second user console includes a fourth arm having a fourth joint and a fourth actuator disposed at the fourth joint, wherein the third actuator is coupled to the first manifold and the fourth actuator is coupled to the second manifold, and wherein the first manifold is in communication with the second manifold such that movements of the third arm about the third joint are mirrored to movements of the fourth arm about the fourth joint and movements of the fourth arm about the fourth joint are mirrored to movements of the third arm about the third joint.

11. A robotic surgical system comprising:
a processing unit;
a surgical robot having a first robot arm; and
a dual user console including:
a first user console in communication with the processing unit and configured to transmit input signals to the processing unit, the processing unit configured to transmit control signals to the surgical robot in response to the input signals such that the surgical robot manipulates the first robot arm in response to the control signals, the first user console including:
a first arm having a first joint;
a first actuator disposed at the first joint and including a first pneumatic cylinder and a first hydraulic cylinder, each of the first pneumatic and hydraulic cylinders configured to actuate the first arm about the first joint and to be back driven by movement of the first arm about the first joint; and
a first manifold coupled to the first pneumatic cylinder and the first hydraulic cylinder; and
a second user console including:
a second arm having a second joint;
a second actuator disposed at the second joint and including a second pneumatic cylinder and a second hydraulic cylinder, each of the second pneumatic and hydraulic cylinders configured to actuate the second arm about the second joint and to be back driven by movement of the second arm about the second joint; and
a second manifold coupled to the second pneumatic cylinder and the second hydraulic cylinder,
wherein the first manifold is in communication with the second manifold such that movements of the first arm about the first joint are mirrored to movements of the second arm about the second joint and movements of the second arm about the second joint are mirrored to movements of the first arm about the first joint.

12. The robotic surgical system according to claim 11, wherein the first user console includes a third arm having a third joint and a third actuator disposed at the third joint, and wherein the second user console includes a fourth arm having a fourth joint and a fourth actuator disposed at the fourth joint.

13. The robotic surgical system according to claim 12, wherein the third actuator is coupled to the first manifold and the fourth actuator is coupled to the second manifold, and wherein the first manifold is in communication with the second manifold such that movements of the third arm about the third joint are mirrored to movements of the fourth arm about the fourth joint, and movements of the fourth arm about the fourth joint are mirrored to movements of the third arm about the third joint.

14. The robotic surgical system according to claim 12, wherein the first user console includes a third manifold disposed on the third arm, and the second user console includes a fourth manifold disposed on the fourth arm, the third manifold in communication with the fourth manifold such that movements of the third arm about the third joint are mirrored to movements of the fourth arm about the fourth joint, and movements of the fourth arm about the fourth joint are mirrored to movements of the third arm about the third joint.

15. The robotic surgical system according to claim 14, wherein the first user console includes a first console manifold in direct communication the first and third manifolds and the second user console includes a second console manifold in direct communication with the second and fourth manifolds, the first and second console manifolds in direct communication with one another such that the first and second manifolds and third and fourth manifolds are in communication with one another.

16. The robotic surgical system according to claim 15, wherein the dual user console include pneumatic and hydraulic console interconnects, the pneumatic console interconnect including a first discreet pneumatic channel configured to couple the first and second actuators and a second discreet pneumatic channel configured to couple the third and fourth actuators, the hydraulic console interconnect including a first discreet hydraulic channel configured to couple the first and second actuators and a second discreet hydraulic channel configured to couple the third and fourth actuators.

17. A method of controlling a surgical robot or a surgical simulator, the method comprising
manipulating a first arm of a first user console about a first joint such that a first actuator associated with the first joint transmits first pneumatic and hydraulic signals to a second actuator associated with a second joint of a second arm of a second user console to actuate the second arm to mirror the manipulation of the first arm; and
manipulating the second arm of the second user console about the second joint such that the second actuator transmits second pneumatic and hydraulic signals to the first actuator associated with the first joint to actuate the first arm to mirror the manipulation of the second arm.

18. The method according to claim 17, wherein manipulating the first arm includes transmitting the first pneumatic and hydraulic signals to a first manifold of the first user console which transmits the first pneumatic and hydraulic signals to a second manifold of the second user interface.

19. The method according to claim 18, wherein the first manifold transmitting the first pneumatic and hydraulic signals to the second manifold includes via wire or wirelessly transmitting the first pneumatic and hydraulic signals.

20. The method according to claim 18, further comprising:
selecting a preprogramed surgical technique such that a processing unit of the surgical robot or the surgical simulator transmits control signals to the first manifold, the first manifold transmitting pneumatic and hydraulic signals to the first actuator in response to the control signals such that the first actuator actuates the first arm about the first joint.
